(19) United States
(12) Patent Application Publication
(10) Pub. No.: US 2022/0016043 A1
(43) Pub. Date: Jan. 20, 2022

(54) METHOD FOR MANUFACTURING RED BLOOD CELL-SHAPED NANOSTRUCTURE USING MULTI-FLUID ELECTROSPRAY METHOD INCLUDING MULTIPLE NOZZLES

(71) Applicants: University-Industry Foundation, Yonsei University, Seoul (KR); Research Cooperation Foundation of Yeungnam University, Gyeongsan-si (KR)

(72) Inventors: Jeong Hoon BYEON, Gyeongsan-si (KR); Jung Ho HWANG, Seoul (KR); Dae Hoon PARK, Seoul (KR); Kang Sik NAM, Gyeonggi-do (KR)

(73) Assignees: University-Industry Foundation, Yonsei University, Seoul (KR); Research Cooperation Foundation of Yeungnam University, Gyeongsan-si (KR)

(21) Appl. No.: 17/296,260

(22) PCT Filed: Nov. 25, 2019
(86) PCT No.: PCT/KR2019/016255

§ 371 Date: May 24, 2021

(30) Foreign Application Priority Data
Nov. 23, 2018 (KR) 10-2018-0146245

(51) Int. Cl. A61K 9/51; C08J 3/12; B05D 1/04

(52) U.S. Cl. CPC A61K 9/5192; A61K 9/5138; C08J 2333/08; B05D 1/04; C08J 3/122

(57) ABSTRACT

The present application relates to a method for manufacturing a red blood cell-shaped nanostructure and a red blood cell-shaped nanostructure manufactured by the manufacturing method thereof. More specifically, the present application relates to a method for manufacturing a red blood cell-shaped nanostructure using a multi-fluid electrospray method including multiple nozzles, and a red blood cell-shaped nanostructure manufactured by the manufacturing method thereof.

### TECHNICAL FIELD

The present application relates to a method for manufacturing a red blood cell-shaped nanostructure and a red blood cell-shaped nanostructure manufactured by the manufacturing method thereof. More specifically, the present application relates to a method for manufacturing a red blood cell-shaped nanostructure using a multi-fluid electrospray method including multiple nozzles, and a red blood cell-shaped nanostructure manufactured by the manufacturing method thereof.

### BACKGROUND ART

The global demand for nanomaterial technology is increasing rapidly, and accordingly, the market also tends to be growing significantly. In the case of South Korea, a budget of more than 70 billion won is allocated for the development of nanomaterials every year, and the markets thereof are expected to grow significantly as various applications using nanoparticles are developed in the future.

In particular, particles used for drugs have different effects on cells depending on their shape, thus changing their efficacy. Red blood cells have a morphological advantage of being able to be uptaken in various tissues (including tumors) in the human body like nano-sized drugs while moving freely and easily within blood vessels despite their relatively large size of about 7 microns. Therefore, some research and development has been performed to mimic red blood cells.

However, a technique for producing generally used red blood cell-mimicking particles in the related art is very complicated because the red blood cell-mimicking particles are produced through a wet etching process that goes through several steps such as isolation and purification. Among them, an example is illustrated in FIG. 1. As illustrated in FIG. 1, an organic solvent such as 1,4-bis(triethoxysilyl)benzene (BTEB) needs to be used, and a high-temperature process exceeding 900° C. is required. Further, after a nanostructure is manufactured such that SiO₂, which serves as a template, is located in the core, that is, inside the nanostructure, a hollow structure is manufactured by removing the template located in the core. Specifically, a complicated process of performing etching using a hydrofluoric acid solution is required.

Therefore, there is a need for research capable of manufacturing a red blood cell-mimicking nanostructure in a one-stop manner without using this complicated process.

### DISCLOSURE

### Technical Problem

An exemplary embodiment of the present application has been made in an effort to provide a red blood cell-shaped nanostructure in a one-stop manner without going through a complicated wet process.

An exemplary embodiment of the present application has also been made in an effort to provide a discoid-shaped or bowl-shaped nanostructure having better cell absorption than a spherical nanostructure in the related art.

An exemplary embodiment of the present application has also been made in an effort to provide a manufacturing method capable of mass-producing particles having a uniform size distribution.

### Technical Solution

An aspect of the present application relates to a method for manufacturing a red blood cell-shaped nanostructure using a multi-fluid electrospray method including multiple nozzles.

As an example, the manufacturing method includes: a step of preparing a gas and a liquid polymer compound; a step of spraying the gas through a first nozzle and the liquid polymer compound through a second nozzle, which is coaxial with the first nozzle and has a diameter larger than a diameter of the first nozzle; and a step of collecting nanostructures sprayed through the first nozzle and the second nozzle, in which a shape of the nanostructure is a red blood cell shape.

As an example, a flow rate ratio of the liquid polymer compound sprayed through the second nozzle and the gas sprayed through the first nozzle is 1:0.1 to 100.

As an example, a range of voltage applied to the first nozzle and the second nozzle is 7 kV to 9 kV.

As an example, the gas is air.

As an example, the polymer compound is a Eudragit-based compound.

As an example, the Eudragit-based compound includes at least one of Eudragit-L, Eudragit-RL, and Eudragit-RS.

As an example, the nanostructure is collected after moving 60 cm to 70 cm.

As an example, a shape of the nanostructure is a core-shell shape during spraying through tips of the first nozzle and the second nozzle, the shell includes a polymer compound, the core includes a gas, but the gas of the core is released through the shell, and a shape of the collected nanostructure is a red blood cell shape.

As an example, the gas further includes at least one of a therapeutic agent, a diagnostic agent and a contrast agent.

As an example, the liquid polymer compound further includes at least one of a therapeutic agent, a diagnostic agent and a contrast agent.

As an example, through a third nozzle, which is coaxial with the first nozzle and the second nozzle and has a diameter larger than a diameter of the second nozzle, a liquid polymer compound different from the liquid polymer compound sprayed through the second nozzle is sprayed in combination.

Another aspect of the present application relates to a red blood cell-shaped nanostructure manufactured by the above-described manufacturing method, in which the nanostructure has a biconcave discoid shape or bowl shape.

As an example, the nanostructure has an average outer diameter of 300 nm to 550 nm and an average inner diameter of 230 nm to 270 nm.

### Advantageous Effects

According to an exemplary embodiment of the present application, a red blood cell-shaped nano structure can be manufactured without going through a composite process using a template in the related art.

According to an exemplary embodiment of the present application, a red blood cell-shaped nanostructure can be manufactured in a one-stop manner at room temperature.

According to an exemplary embodiment of the present application, a nanostructure having a uniform size and shape can be manufactured using an electrospray method.

According to an exemplary embodiment of the present application, a nanostructure which is harmless to the human body can be manufactured using a bio-friendly polymer.

According to an exemplary embodiment of the present application, a nanostructure having excellent cell absorbability can be manufactured.

According to an exemplary embodiment of the present application, a nanostructure including various therapeutic agents, diagnostic agents, and contrast agents can be manufactured.

According to an exemplary embodiment of the present application, the nanostructure can be variously applied to devices which require hollow particles, such as various energy devices such as a battery, a supercapacitor, a solar cell, and a fuel cell.

### DESCRIPTION OF DRAWINGS

FIG. 1 is a view schematically illustrating one of the techniques for manufacturing a core-shell nanostructure in the related art.

FIG. 2 is a flow-chart of a method for manufacturing a red blood cell-shaped nanostructure, which is an exemplary embodiment of the present application.

FIG. 3 is a schematic view for describing a method for manufacturing a red blood cell-shaped nanostructure, which is an exemplary embodiment of the present application.

FIG. 4 is a schematic view for describing an electrospray method in the method for manufacturing a red blood cell-shaped nanostructure, which is an exemplary embodiment of the present application.

FIG. 5 is a schematic view of a biconcave discoid-shaped nanostructure, which is an exemplary embodiment of the present application.

FIG. 6 is a schematic view of a biconcave bowl-shaped nanostructure, which is an exemplary embodiment of the present application.

FIGS. 7A to 7C are scanning electron microscope (SEM) images for a red blood cell-shaped nanostructure, a transitional nanostructure, and a spherical nanostructure, respectively.

FIG. 8 is a graph of results derived by a fluorescent activated cell sorter (FACS) for each of a control, a spherical nanostructure, a transitional nanostructure and a red blood cell-shaped nanostructure.

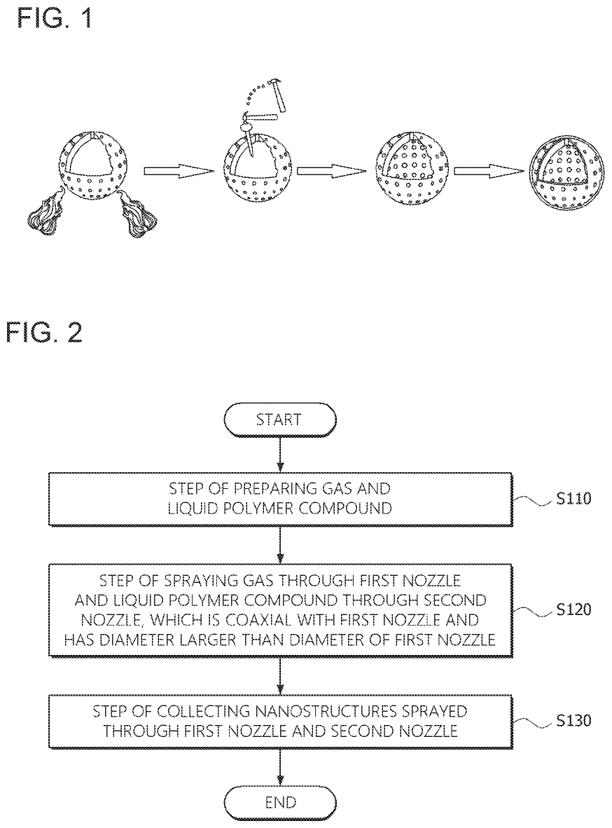

### MODES OF THE INVENTION

The terms used in the present application are used only to describe specific embodiments, and are not intended to limit the present invention. Singular expressions include plural expressions unless the context clearly indicates otherwise. In the present application, terms such as “include” or “have” are intended to specify the presence of the features, components, and the like described in the specification, and does not mean that one or more other features, components, or the like are not present or cannot be added.

Unless defined otherwise, all terms used herein, including technical or scientific terms, have the same meaning as commonly understood by a person with ordinary skill in the art to which the present invention pertains. Terms such as those defined in commonly used dictionaries should be interpreted as having a meaning consistent with the meaning in the context of the relevant art and should not be interpreted in an ideal or overly formal sense unless explicitly defined in the present application.

In the present application, the term “nano” may refer to a size in nanometers (nm), and may refer to, for example, a size of 1 to 1,000 nm, but is not limited thereto. Further, in the present specification, the term “nanoparticles” may refer to particles having an average particle diameter of a nanometer (nm) unit, and may refer to, for example, particles having an average particle diameter of 1 to 1,000 nm, but is not limited thereto.

Hereinafter, a method for manufacturing a red blood cell-shaped nanostructure of the present application will be described in detail with reference to the accompanying drawings. However, the accompanying drawings are exemplary and the scope of the red blood cell-shaped nanostructure of the present application and a nanostructure manufactured using the same is not limited by the accompanying drawings.

FIG. 2 is a flow-chart of a method for manufacturing a red blood cell-shaped nanostructure, which is an exemplary embodiment of the present application.

As illustrated in FIG. 2, the method for manufacturing a red blood cell-shaped nanostructure of the present application includes a step of preparing a gas and a liquid polymer compound (S110), a step of spraying the gas through a first nozzle and the liquid polymer compound through a second nozzle, which is coaxial with the first nozzle and has a diameter larger than a diameter of the first nozzle (S120) and a step of collecting nanostructures sprayed through the first nozzle and the second nozzle (S130).

Hereinafter, the present application will be described in more detail for each step.

First, a gas and a liquid polymer compound are prepared (S110).

The gas is not particularly limited, and it is preferred to use general air present in the atmosphere due to excellent economic feasibility. As described below, the structure sprayed through the nozzle has a shape similar to a core-shell, and in this case, air occupies the core, but the air escapes through the pores of the shell and the like. For example, after the inside of the nanostructure is inflated by flowing air to an inner nozzle using a double nozzle electrospray method, a red blood cell-shaped nanostructure may be finally manufactured while the air escapes.

In addition, the liquid polymer compound is not particularly limited, but preferably, the liquid polymer compound is a Eudragit-based compound. Furthermore, more preferably, the Eudragit-based compound includes at least one of Eudragit-L, Eudragit-RL, and Eudragit-RS.

Eudragit-L is a copolymer in which methacrylic acid and methyl methacrylate are included at a ratio of 1:1. Eudragit-RS and Eudragit-RL are compounds in which ethyl acrylate, methyl methacrylate and methacrylic acid ester are included along with quaternary ammonium groups, and may be represented by the following Chemical Formula. In the following Chemical Formula, m=0.2, n=2 and o=1 indicate Eudragit-RL, and m=0.1, n=2 and o=1 indicate Eudragit-RS.

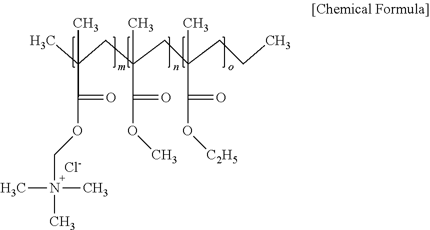

Moreover, both the air and the liquid polymer compound are sprayed through a first nozzle and a second nozzle which is coaxial with the first nozzle and has a diameter larger than a diameter of the first nozzle, respectively (S120).

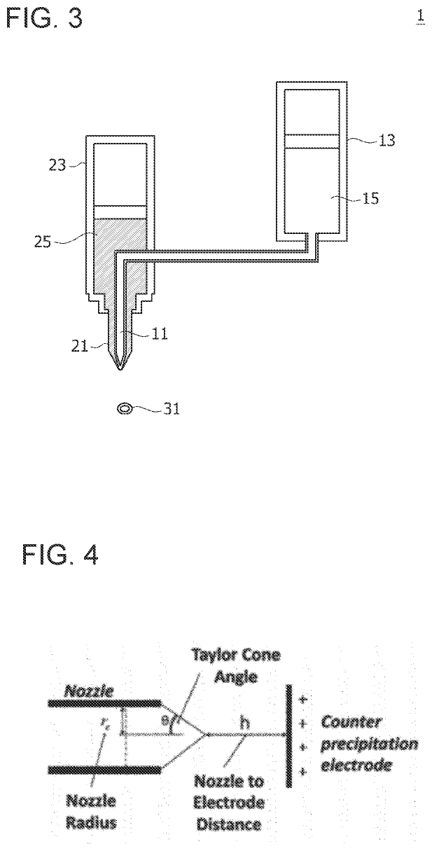

The spraying uses an electrospray method, and FIG. 3 illustrates a schematic view of an electrospray device for describing a method for manufacturing a red blood cell-shaped nanostructure, which is an exemplary embodiment of the present application.

As illustrated in FIG. 3, an electrospray device 1 according to an exemplary embodiment of the present application includes a double nozzle. A gas supply unit 13 is filled with a gas, and the gas is sprayed through a first nozzle 11. Further, a second nozzle 21 is coaxial with the first nozzle 11 and has a diameter larger than a diameter of the first nozzle 11. A liquid polymer compound supply unit 23 is filled with a liquid polymer compound, and the liquid polymer compound is sprayed through the second nozzle 21. Through this, a red blood cell-shaped nanostructure 31 may be obtained.

In the electrospray method, a force for spraying liquid droplets in an electrospray device allows a solution having a suitable electrical conductivity to pass through a nozzle to which a high voltage is applied, so that anions move toward a nozzle that acts as a positive electrode due to the attractive force, and cations dissolved in a liquid move toward the curved surface of a liquid due to the repulsive force. For a liquid present on the curved surface of the liquid, liquid droplets are not produced because the surface tension of the liquid initially acting on the curved surface of the liquid is larger than the electric force, but when a voltage applied to the nozzle is increased, a cone-shaped liquid curved surface is formed on the tip of the nozzle, which is called a Taylor cone.

FIG. 4 is a schematic view for describing a Taylor cone by an electrospray method in the method for manufacturing a red blood cell-shaped nanostructure, which is an exemplary embodiment of the present application.

As illustrated in FIG. 4, an ultrafine liquid column is formed by receiving surface shear stress resulting from an electric force at the end of the Taylor cone by the applied voltage, and then a breakup phenomenon acting on the surface of the liquid column appears.

In the present application, in the case of a double nozzle, a gas is sprayed through the inner nozzle and a liquid polymer compound is sprayed through the outer nozzle using multiple nozzles. Through this, using a two-fluid electrospray technique, a bio-friendly polymer is inflated through an effect of filling a balloon with air, and then a red blood cell-shaped nanostructure having a size of approximately 400 nm may be finally manufactured while the air escapes.

As described above, the electrospray method is a technique for forming a strong electric field when a predetermined voltage or more is applied between a nozzle and a substrate, forming a Taylor cone when an electrostatic repulsive force overcomes surface tension in a solution due to the formation of the strong electric field, and producing nanoparticles, and in this case, the characteristics (size, shape) of nanoparticles to be produced may be variously controlled by adjusting the viscosity, surface tension, applied voltage, flow rate, and the like of the solution. Electrospraying is a method suitable for mass production of nanoparticles, and has an advantage in that particles can be simply and easily produced by applying a high voltage, and the size and shape of the produced particles are very uniform.

In the present application, a flow rate ratio of the liquid polymer compound sprayed through the second nozzle and the gas sprayed through the first nozzle is preferably 1:0.1 to 10.

However, the flow rate of the liquid polymer compound may be 1 to 20 μlpm, preferably 10 μlpm, and the flow rate of the gas is preferably 1 to 10 μlpm.

When the flow rate of the liquid polymer compound is increased, since the size of particles is gradually increased, the size of the nanostructure targeted by the present application can be adjusted by controlling such a flow rate. In the present application, the flow rate of the liquid polymer compound may be fixed in order to manufacture a nanostructure having a size of several hundred nanometers (400 nm or less).

Further, a range of voltage applied to the first nozzle and the second nozzle is preferably 7 kV to 9 kV. This is a voltage range in which a Taylor cone is formed. When a voltage to be applied is less than 7 kV, it is difficult to form liquid droplets, and when a voltage to be applied is more than 9 kV, a multi-jet is formed, so that a desired form cannot be obtained.

Then, nanostructures sprayed through the first nozzle and the second nozzle are collected (S130).

It is preferred that the nanostructures are collected after moving 60 to 70 cm. When the moving distance becomes too short, the nanostructure is electrospun rather than electrosprayed, and thus is produced in the form of a fiber rather than liquid droplets.

As described above, a shape of the nanostructure is a core-shell shape during spraying through tips of the first nozzle and the second nozzle, the shell includes a polymer compound, the core includes a gas, but the gas of the core is released through the shell, and a shape of the collected nanostructure is changed into a red blood cell shape.

In addition, at least one of a therapeutic agent, a diagnostic agent and a contrast agent may be further included in the gas sprayed through the first nozzle. In this case, as described above, even though the gas escapes out of the polymer compound layer, the therapeutic agent, the diagnostic agent and the contrast agent remain inside a final red blood cell-shaped nanostructure. Therefore, these nanostructures may act as medical material carriers for therapeutic purposes, diagnostic purposes, and the like, depending on the purpose of the present application.

In addition, the liquid polymer compound may further include at least one of a therapeutic agent, a diagnostic agent and a contrast agent. In this case, the therapeutic agent, the diagnostic agent and the contrast agent also remain in the polymer compound layer itself of the final red blood cell-shaped nanostructure. Therefore, these nanostructures may also act as medical material carriers for therapeutic purposes, diagnostic purposes, and the like, depending on the purpose of the present application.

That is, after a nanostructure is manufactured, a medical material may also be carried, but when the nanostructure is manufactured, a therapeutic agent, a diagnostic agent and a contrast agent may be mixed, sprayed, and thus used as a medical material carrier intended by the present application.

Furthermore, through a third nozzle, which is coaxial with the first nozzle and the second nozzle and has a diameter larger than a diameter of the second nozzle, a liquid polymer compound different from the liquid polymer compound sprayed through the second nozzle may be sprayed in combination. In this case, three materials may be combined. For example, when a gas is sprayed through the first nozzle, a first liquid polymer compound is sprayed through the second nozzle, and a second liquid polymer compound is sprayed through the third nozzle simultaneously, a nanostructure having a polymer compound layer formed of a double layer may be manufactured unlike the nanostructure described above.

In this case, a therapeutic agent, a diagnostic agent and a contrast agent may also be mixed with at least one of the gas, the first liquid polymer compound and the second liquid polymer compound, and the resulting mixture may be sprayed, and thus may be used as a medical material carrier intended by the present application.

Another aspect of the present application is a red blood cell-shaped nanostructure manufactured by the above-described manufacturing method.

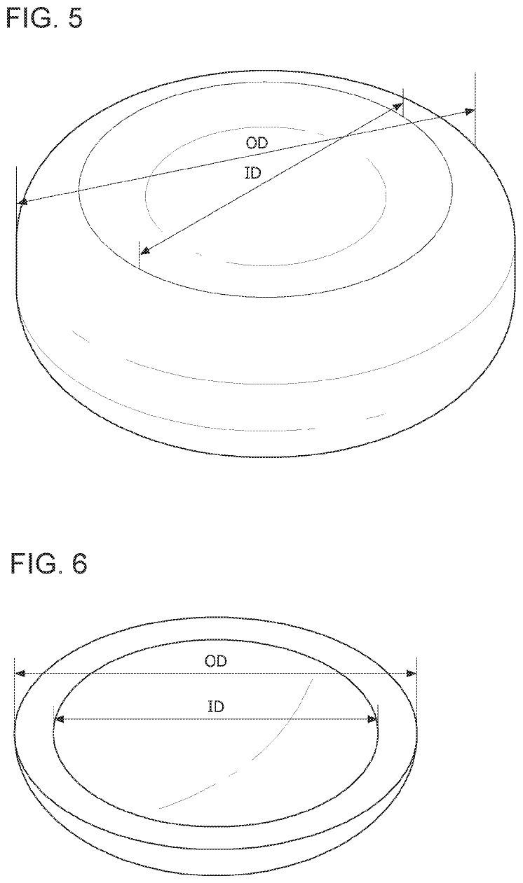

FIG. 5 illustrates a schematic view of a biconcave discoid-shaped nanostructure, which is an exemplary embodiment of the present application, and FIG. 6 illustrates a schematic view of a biconcave bowl-shaped nanostructure, which is an exemplary embodiment of the present application.

As described above, the shape of the red blood cell nanostructure may be a biconcave discoid shape. In this case, both (the top and the bottom) have a concave structure. Furthermore, as illustrated in FIG. 5, the shape of the red blood cell nanostructure may be a biconcave bowl shape. In this case, only one side has a biconcave structure.

The nanostructure may have an average outer diameter (OD) of 300 to 550 nm and an average inner diameter (ID) of 230 to 270 nm. When the size of the nanostructure is too large, the nanostructure may be ingested by phagocytosis inside the cell against the intention of the present application, and when the size of the nanostructure is too small, the nanostructure may disappear.

Hereinafter, the present application will be described in more detail through an Experimental Example.

### Experimental Examples

The following experiments were performed to confirm whether a red blood cell-shaped nanostructure of the present application could be manufactured. First, Example 1 was manufactured as follows. An electrospray device having the above-described double nozzle was used, and air was sprayed through an inner nozzle and Eudragit-RS (concentration: 800 mg/10 mL) was sprayed through an outer nozzle. In this case, the flow rate of the air was 5 μlpm, and the flow rate of Eudragit-RS was μlpm. A voltage of 8 kV was applied to the nozzles, and the distance from a collector was 65 cm.

Further, Example 2 was manufactured as follows. An electrospray device having the above-described double nozzle was used, and air was sprayed through an inner nozzle and Eudragit-RS (concentration: 400 mg/10 mL) was sprayed through an outer nozzle. In this case, the flow rate of the air was 5 μlpm, and the flow rate of Eudragit-RS was 10 μlpm. A voltage of 8 kV was applied to the nozzles, and the distance from a collector was 65 cm.

In addition, Comparative Examples 1 and 2 were manufactured as follows. Comparative Example 1 was manufactured as follows. An electrospray device having the above-described double nozzle was used, and air was sprayed through an inner nozzle and Eudragit-RS (concentration: 100 mg/10 mL) was sprayed through an outer nozzle. In this case, the flow rate of the air was 5 μlpm, and the flow rate of Eudragit-RS was 10 μlpm. A voltage of 8 kV was applied to the nozzles, and the distance from a collector was 65 cm.

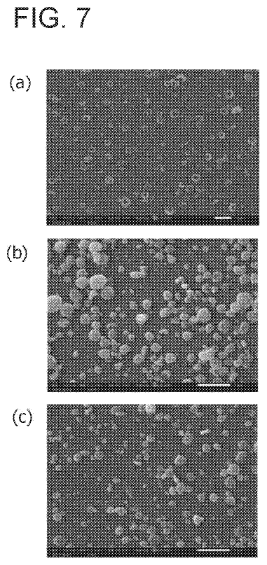

In Comparative Example 2, as a control, phosphate buffered saline (PBS) (a buffer solution) was simply used without including the particles as described above. A SEM image for a red blood cell-shaped nanostructure (Example 1), a transitional nanostructure (Example 2), and a spherical nanostructure (Comparative Example 1) are illustrated in FIGS. 7A to 7C, respectively. As illustrated in FIGS. 7A to 7C, Example 1 is a red blood cell-shaped nano structure, whereas Comparative Example 1 is spherical.

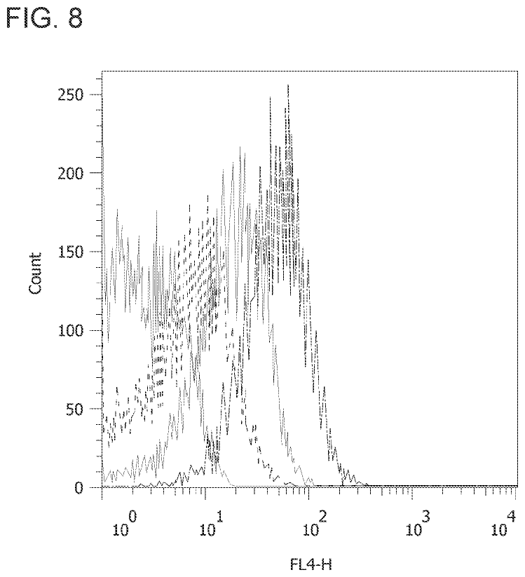

In addition, an experiment for determining how well a drug was absorbed (cellular uptake) by the cell membrane was additionally performed on Examples 1 and 2 and Comparative Examples 1 and 2. For this purpose, a method referred to as fluorescent activated cell sorting (FACS) was used. The FACS is an experimental method using, particularly, an optical principle in flow cytometry, and can confirm how much of the drug is absorbed using a laser when particles and cells in an emulsion state pass through a certain detection area for quick measurement. The results are shown in Table 1 and FIG. 8.

TABLE 1

| | | | Mean: | Color of |
|---|---|---|---|---|
| | | Count | FL4-H | FIG. 8 |
| Example 1 | Red blood cell | 8867 | 57.3 | Green |
| | shape | | | |
| Example 2 | Incomplete red | 9187 | 21.9 | Orange |
| | blood cell shape | | | |
| Comparative | Spherical | 8449 | 9.40 | Blue |
| Example 1 | | | | |
| Comparative | control | 8469 | 3.52 | Red |
| Example 2 | | | | |

As shown in Table 1 and FIG. 8, it could be confirmed that the peaks of green and orange colored graphs were high, and it could be confirmed that in the case of Example 1, the absorption capacity was about 6-fold higher than that of Comparative Example 1.

Although the present application has been described above with reference to preferred embodiments of the present application, it is to be understood by those skilled in the art that the present application can be variously modified and changed within the scope not departing from the spirit and scope of the present invention described in the following claims.

### DESCRIPTION OF REFERENCE NUMERALS AND SYMBOLS

1: Electrospray device
11: First nozzle
13: Air supply unit
21: Second nozzle
23: Liquid polymer compound supply unit
25: Liquid polymer compound
31: Red blood cell-shaped nanostructure

1. A method for manufacturing a red blood cell-shaped nanostructure using a multi-fluid electrospray method comprising multiple nozzles, the method comprising steps of:
preparing a gas and a liquid polymer compound;
spraying the gas through a first nozzle and the liquid polymer compound through a second nozzle, which is coaxial with the first nozzle and has a diameter larger than a diameter of the first nozzle; and
collecting nanostructures sprayed through the first nozzle and the second nozzle,
wherein a shape of the nanostructure is a red blood cell shape.

2. The method of claim 1, wherein a flow rate ratio of the liquid polymer compound sprayed through the second nozzle and the gas sprayed through the first nozzle is 1:0.1 to 10.

3. The method of claim 1, wherein a range of voltage applied to the first nozzle and the second nozzle is 7 kV to 9 kV.

4. The method of claim 1, wherein the gas is air.

5. The method of claim 1, wherein the polymer compound is a Eudragit-based compound.

6. The method of claim 5, wherein the Eudragit-based compound comprises at least one of Eudragit-L, Eudragit-RL, and Eudragit-RS.

7. The method of claim 1, wherein the nanostructure is collected after moving 60 cm to 70 cm.

8. The method of claim 1, wherein a shape of the nanostructure is a core-shell shape during spraying through tips of the first nozzle and the second nozzle, the shell comprises a polymer compound, the core comprises a gas, but the gas of the core is released through a shell, and a shape of the collected nanostructure is a red blood cell shape.

9. The method of claim 1, wherein the gas further comprises at least one of a therapeutic agent, a diagnostic agent and a contrast agent.

10. The method of claim 1, wherein the liquid polymer compound further comprises at least one of a therapeutic agent, a diagnostic agent and a contrast agent.

11. The method of claim 1, wherein through a third nozzle, which is coaxial with the first nozzle and the second nozzle and has a diameter larger than a diameter of the second nozzle, a liquid polymer compound different from the liquid polymer compound sprayed through the second nozzle is sprayed in combination.

12. A red blood cell-shaped nanostructure manufactured by the manufacturing method of claim 1,
wherein the nanostructure has a biconcave discoid shape or bowl shape.

13. The red blood cell-shaped nanostructure of claim 12, wherein the nanostructure has an average outer diameter of 300 nm to 550 nm and an average inner diameter of 230 nm to 270 nm.
